(19) United States
(12) Patent Application Publication
(10) Pub. No.: US 2005/0090860 A1
(43) Pub. Date: Apr. 28, 2005

(54) Segmented plug for tissue tracts

(76) Inventor: Loran James Paprocki, St. Louis Park, MN (US)

Correspondence Address: David E. Bruhn;DORSEY & WHITNEY LLP, Intellectual Property Department, 50 South Sixth Street, Suite 1500, Minneapolis, MN, 55402-1498, US

(21) Appl. No.: 10/692,277

(22) Filed: Oct. 23, 2003

(51) Int. Cl. A61B 17/08

(52) U.S. Cl. 606/213

(57) ABSTRACT

A tract plug of a selected number of individual plug segments of biocompatible material, wherein the segments are separably connected so that the overall length of the tract plug may be selected by separating one of more segments from a remaining segment or segments. The invention encompasses a method of joining and separating a selected number of individual segments of biocompatible material, and a method of using a tract plug comprising a selected number of individual segments of biocompatible material.

### BACKGROUND

The present invention relates to wound closure and healing devices, and methods for closing, packing or healing wounds. More particularly, the invention relates to a collagen plug for facilitating hemostasis in a wound, for example in a catheterization tract, core or other puncture wound.

Medical procedures which create and leave tissue tracts, such as catheterization and biopsy procedures, are very common. Catheterization procedures are becoming ever more common as the advantages of minimally invasive surgical procedures are increasingly recognized and acknowledged.

As the numbers of catheterizations have increased, particularly those involving vascular procedures, devices to close the wounds have proliferated as well. For example, U.S. Pat. No. 6,179,863 (Kensey et al.) and U.S. Pat. No. 5,411,520 (Nash et al.), the teachings and disclosures of which are incorporated herein by reference, disclose hemostatic puncture closure systems and methods. Referring to the Nash et al. patent, the disclosed system involves a collagen plug sealing member which is positioned in a puncture tract immediately adjacent to a blood vessel punctured during a procedure. A suitable hemostatic resorbable material such as a collagen sponge or foam is used to make the plug. The plug may be formed of compressed collagen or other suitable hemostatic material so that it expands in the presence of blood and/or body fluid within the tract. One suitable plug comprises a cylindrical member formed of a compressible resorbable collagen foam, such as that sold by Kinsey Nash of Exton, Pa. The plug is moved into the tract and then a filament/pulley arrangement is manipulated to position the plug next to the blood vessel. After disposition, the plug is deformed within the tract by tamping. Thus, the plug is lodged within the tract immediately adjacent to the puncture in the vessel.

U.S. Pat. No. 6,440,153 (Cragg et al.) discloses a device and method for facilitating hemostasis of a biopsy tract. The system includes an adaptor and a syringe for delivering an absorbable sponge in a hydrated state into the wound. The adaptor includes a tapered lumen for hydrating and compressing a relatively absorbable sponge for delivery through a relatively small cannula. The sponge is injected through a needle into the biopsy tract using fluid, or it may be delivered to the needle by fluid and delivered to the tract by a plunger or stylet. The sponge material is biosorbable. While the state of the sponge is manipulated, i.e., the sponge is hydrated, and it may have a particular shape, e.g., increased proximal cross section areas; at the end of the procedure the sponge (or plug or pledget) is entirely within the tract. In an alternative embodiment of the invention, it is disclosed that the pledget may be delivered into the tract in an elongated trail to fill the entire tract. One method involves the delivery of a pledget into a needle by a predetermined amount of fluid and withdrawal of the needle at a selected velocity while the material is ejected. The trail may extend along the entire biopsy tract to or past a surface of an organ. As an alternative to delivery of a pledget as a trail, it may be delivered as a plug, or a combination of delivery of plugs and trails may be used. The patent discloses that the pledget material may be delivered within a single anatomical structure or may cross two or more anatomical structures such as an organ, surrounding tissue or the facial layer. The patent discloses that it may be desirable to deliver multiple pledgets in spaced apart positions along a tract, and that the sponge material must be cut to a particular size. There is no discussion about providing a plug which is adapted to be used in tracts of various lengths without being in liquid or substantially liquid form, or without being cut.

U.S. Pat. No. 6,325,789 (Janzen et al.) discloses a device for inserting a hemostatic material through a tissue channel and placing it against the outside wall of a blood vessel. The hemostatic material is placed in a sheath and advanced through the sheath to near the vessel wall. One embodiment of the disclosed invention teaches the use of a plug, preferably of a fibrous collagen material. A special device comprising a sheath and a plug pusher or piston is used to insert the plug. The sheath is inserted through tissue until its leading end is near or abuts the outer wall of the blood vessel and the plug is advanced through the sheath by use of the plug pusher until it abuts the vessel wall. The sheath and pusher are then removed. FIGS. 12a-e show forms of the plug which may be used. The plug may be of any resorbable material, including collagen. The patent discloses the physical form of the plug, for example FIG. 12a shows “loose fibrous material, somewhat like fleece or absorbent cotton or oxygenated cellulous.” Alternatively, a plug of more densely packed material could be used or, as shown in FIG. 12c, one end of the plug might be formed of loose fibrous material and the other could be made of a more densely packed fibrous material. Another type of plug is shown in FIG. 12b wherein one end is a collagen membrane and the other is an expandable collagen sponge. There is no disclosure or teaching about how a plug may be adapted for use in tracts of various lengths other than by cutting. In one embodiment, two plugs are used to fill all of a channel extending from a blood vessel to the skin line. One of the plugs “may be made longer than necessary to reach the skin line, in which case it could be then cut off flush with the skin.” A single plug, the size of the two plugs, could be used instead of two separate plugs. There is no teaching or suggestion about how to avoid the need to cut the plug, nor how to facilitate adjusting a plug to a tract of a certain length without cutting it.

### SUMMARY

One embodiment of the present invention is a tract plug made up of a selected number of individual plug segments of biocompatible material, wherein the segments are separably connected so that the overall length of the tract plug may be selected by separating one of more segments from a remaining segment or segments. The invention encompasses a method of joining and separating a selected number of individual segments of biocompatible material, and a method of using a tract plug comprising a selected number of individual segments of biocompatible material.

One embodiment of the present invention is a tissue tract plug comprised of a selected number of individual segments of biosorbable material, wherein the segments are separably linked so that the overall length of the tract plug may be selected by separating one of more segments from the remaining segment or segments without cutting. The invention encompasses a method of joining a selected number of individual segments of biosorbable material, and a method of using a tract plug comprising a selected number of individual segments of biosorbable material.

One embodiment of the present invention comprises a tract plug comprised of segments of biosorbable material. In one embodiment, the segments are separably linked or connected, in some embodiments across a suitable line of weakness, and in other embodiments by being mechanically and separably meshed at the point at which they abut. In some embodiments, the line of weakness comprises perforations.

One embodiment of the present invention is a generally elongated body comprising a hemostasis promoting material, wherein the body comprises a selected number of units comprising a hemostasis promoting material, wherein the units are separably joined so that the overall length of the body may be selected initially by joining a selected number of the units and subsequently by separating or detaching one or more of the joined units from each other or the remaining units. The invention encompasses a method of joining a selected number of units comprising a hemostasis promoting material, and a method of using a body comprising a selected number of the units.

One advantage of the present invention is that it can be used for facilitating closure or packing and healing of wounds, including tissue tracts, after trauma or medical procedures. Another advantage is that the plug of the present invention can be used in wounds of various lengths without premeasuring and without requiring cutting of the plug.

One embodiment of the present invention comprises a fibrous collagen plug which is a composite or assembly of a number of smaller fibrous collagen plugs or plug segments in abutting, end-to-end contact with each other. The segments may be similar or dissimilar, either entirely or in part. In some embodiments, the collagen may initially be physically separate segments placed end-to-end, whereby the fibers at one plug segment end become entangled with the fibers at another plug segment end. In other embodiments, the plug may include a superficial, frangible, tearable or breakable attachment between segments.

In some embodiments, when inserted in the tissue tract, some or none of the collagen segments may appear above skin level. Any segments appearing entirely or partially above skin level may be removed by breaking or tearing them away from the segments remaining below skin level. Advantages are having an adjustable length of collagen available to be adapted to tracts of various depths without requiring a physician or physician's assistant to measure or cut the collagen to a selected length.

In one embodiment, the present invention comprises a tract plug adapted for use in tracts of various lengths, wherein the plug comprises a plurality of segments of biosorbable material, e.g., collagen, wherein the segments are separably joined. In one embodiment, the segments are linked across a perforated line of weakness. In use, a segment extending from a tract after placement of the plug in the tract is simply torn off along the line of weakness and discarded while the collagen necessary to provide homeostasis remains within the tract. An advantage is this eliminates the need to use a surgical instrument or other device to cut the exposed collagen. Another advantage is that no depth marker is required on the plug or on the device used to place the plug within the tract.

In one embodiment, the present invention is a method of plugging a tract or wound, comprising the steps of providing a tract plug comprising a number of segments wherein the segments are separable without cutting, inserting the tract plug into the tract, and removing any segment remaining outside the tract, entirely or in part, after the plug is inserted.

In one embodiment, the present invention comprises a delivery device for delivering a tract plug. The delivery device, in one embodiment, comprises a body having an lumen near one end, the end which would be inserted in the tract. The device also includes a plunger, coaxial with the lumen for pushing against one end of abutted collagen segments in the lumen to expel them from the lumen into the tract. As the plunger is depressed, the body of the device is removed from the tract while the collagen is being ejected into the tract. Following the deposit of the collagen, i.e., its dispensing or injection into the tract, any segment or segment portion remaining outside the skin may be removed by simply separating that segment from the next segment in the tract.

In one embodiment, the present invention comprises a device for delivering a segmented collagen plug into a tissue tract to effect hemostasis, wherein individual units or segments of the segmented collagen plug may be removed, without cutting, to appropriately adjust the collagen plug to the length of the tissue tract.

In one embodiment, the present invention comprises a collagen tract plug in three pieces, wherein the pieces are placed end-to-end and compressed, including longitudinally compressed, whereby the two end segments adhere or tend to adhere to the middle segment. The segments may be compressed before or by being inserted into a tract delivery device whereby the collagen plug may be delivered into a tract. In use, the compressed collagen is expanded within the tract by the presence of blood. In some embodiments, the delivery device has a body and a structure for receiving the plug segments; both may be sized to hold a certain length of collagen, e.g., a selected number of plug segments, and to insert it into a tract by urging it into the tract by means of a plunger-like member. In some embodiments, the distance traveled by the plunger-like member within the delivery device is equal to the length of the tract or the distance required to fully insert the collagen into the tract. The body of the delivery device and the structure for receiving may be adapted for use with various types of plugs of hemostasis promoting material, including plugs of varying lengths, diameters and shapes, and those comprised of materials other than collagen.

In some embodiments, the plug material can be selected from materials appropriate to facilitate hemostasis. The makeup or composition of the particular material selected for use for forming the plug may be of any suitable makeup or composition. Similarly, the physical state of the plug prior to, during or after insertion in a tract may be varied depending on the use or desired characteristics. Such states may include, for example, being fibrous, comprised of thick fibers or filaments forming an interlocking or interwoven web or matrix, or of relatively fine fibers forming a fibrous body. A fibrous material with strata or other structures within the body of the plug may be used. Similarly, the physical state may be varied, for example, it may be sponge-like, and the material selected may be brittle, sticky or friable in certain portions or regions or overall.

In some embodiments, the delivery device may be adapted to the type of plug material being used and to its selected physical state, as well as to a particular patient, wound, tract or tract or wound characteristic. For example, the delivery device, and the plug, may be of various shapes and diameters and, the length of the lumen of the delivery device may be varied to accommodate plugs of selected number of segments or selected lengths.

In some embodiments, a procedure in accordance with the present invention may involve starting with a selected number of separate pieces of collagen, in some embodiments three pieces of equal size, wherein the pieces are contacted and crushed or compressed together end-to-end to achieve a plug of a selected length, in some embodiments, generally equal to the length of a tissue tract. In one embodiment, the “crushing” would take place just prior to loading the selected number of plugs into the lumen of a delivery device. In other embodiments, it could take place during the loading as the separate segments are loaded and come into contact with each other, or it could take place after the loading or during delivery of the plug into a tract. The longitudinal “crushing” or compression causes the separate segments to become separably joined, intermeshed and/or friction fit together at adjoining surfaces. In some embodiments, particularly fibrous embodiments, the fibrous surfaces and fibers at the ends of the separate plugs are entangled in a fashion similar to the connection provided by what are commonly known as hook and loop fabrics or connectors.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

FIG. 1, including FIGS. 1a-d, depict embodiments of the present invention, and provide a representation of a delivery device and the steps and methods encompassed by the present invention.

FIG. 2 depicts a tract plug in accordance with the present invention.

FIG. 3 depicts a tract plug in accordance with the present invention.

FIG. 4 depicts a tract plug in accordance with the present invention.

FIG. 5 depicts one embodiment of the tract plug delivery device in accordance with the present invention, with an embodiment of a tract plug in a lumen of the device ready to be ejected into a tract

FIG. 6 depicts the delivery device depicted in FIG. 5 with the collagen as it would appear ejected into a tract.

FIG. 7 depicts another embodiment of the plug of the present invention.

FIG. 8 depicts another embodiment of the plug of the present invention.

FIG. 9 depicts another embodiment of the plug of the present invention.

### DETAILED DESCRIPTION

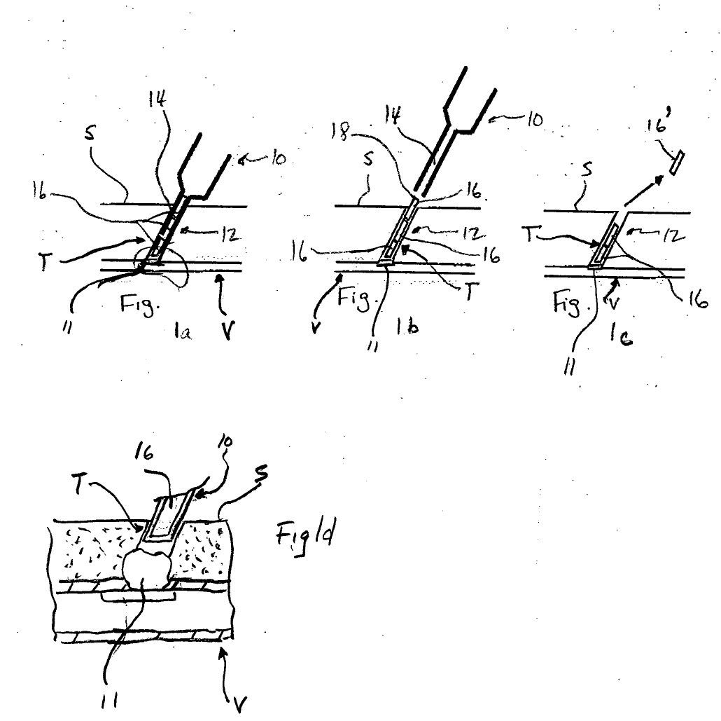

Referring to the accompanying figures, FIGS. 1a-c depict embodiments of the present invention. FIG. 1a depicts a syringe-like delivery device 10 in schematic representation extending into a tract “T” in preparation for delivering the plug 12 in the lumen 14 of the device into the tract. The device 10 is depicted loaded with a tract plug 12 in accordance with the present invention. As depicted in more detail in FIG. 1d (drawn from the circled area in FIG. 1a), the device 10 is placed or inserted in the tract to the extent that its tip and one end of the tract plug 12 are adjacent to, abutting or nearly abutting a previously deployed vascular closure device 11, such as those closure devices known in the art and suitable for closing an opening in a vessel “V.”

FIG. 1b depicts the delivery of the plug 12 which, in this instance, comprises three individual plug segments or units 16, after it is ejected from the delivery device 10 into the tract “T”. Note that the tract is shorter than the overall body or length of the plug 12, i.e., one end 18 of one of the plug segments 16 extends above the skin level “S” of the patient. Note also that, while three segments 16 are depicted, more or fewer may comprise the plug 12. The selected number of segments or units 16 may be based on tract length, tract width, user preference, anatomical variables, the performance characteristics of the material forming the plug 12 and/or plug segments 16, and other factors.

Turning to FIG. 1c, one advantage of the segmented plug body 12 of the present invention is depicted. Namely, because, as shown in FIG. 1b, the plug body 12 is longer than the tract, it is simply and conveniently adjusted, without cutting, by separating one of the plug segments 16, the segment 16′ protruding from the tract above the skin level, from the other individual segments 16 remaining in the tract.

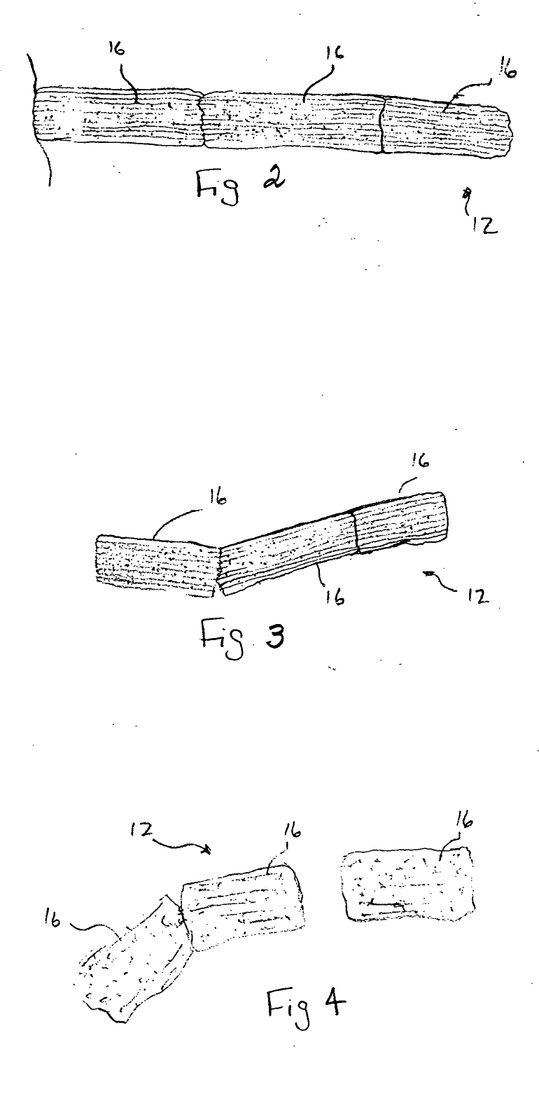

FIGS. 2-4 depict embodiments of the tract plug 12 of the present invention. Referring to FIG. 2, the tract plug 12 comprises three individual segments or units 16 of a biosorbable, hemostasis promoting material, for example, collagen. Other suitable materials or compositions which may be used in accordance with the present invention include Gelfoam, manufactured by Upjohn, other commercially available biocompatible materials, suitable hemostasis promoting materials, materials incorporating fibrin glue and thrombin, et. The plug 12 depicted in FIG. 3 comprises three individual member units or segments 16 of collagen, in this instance, a fibrous collagen. However, other suitable forms and states of collagen or other hemostasis promoting material may be used. Also, the physical state of the selected hemostasis promoting material may be selected from suitable physical states. For example, it may be of a sponge-like consistency, it may be fibrous as depicted, or it may be generally fungible or formed of a particle-like consistency. Similarly, the relative size and shape of the entire plug body 12 and of the individual segments 16 can be selected. They may be relatively longer or shorter, and they may assume any configuration. For example, they may be other than cylindrical, and the overall plug body 12 may assume a tapered shape wherein any segment 16 has a larger outer diameter or a tapered shape.

FIGS. 2, 3 and 4 depict the separable nature of the segments 16 comprising the tract plug body 12, namely, that one or more of the individual segments 16 can be separated from each other to shorten the overall length of the plug body 12. In depicting this, they also depict one embodiment of a method of using the plug of the present invention, particularly the step or sequence of separating one (or more) segments of the plug 12 to, for example, adjust its length.

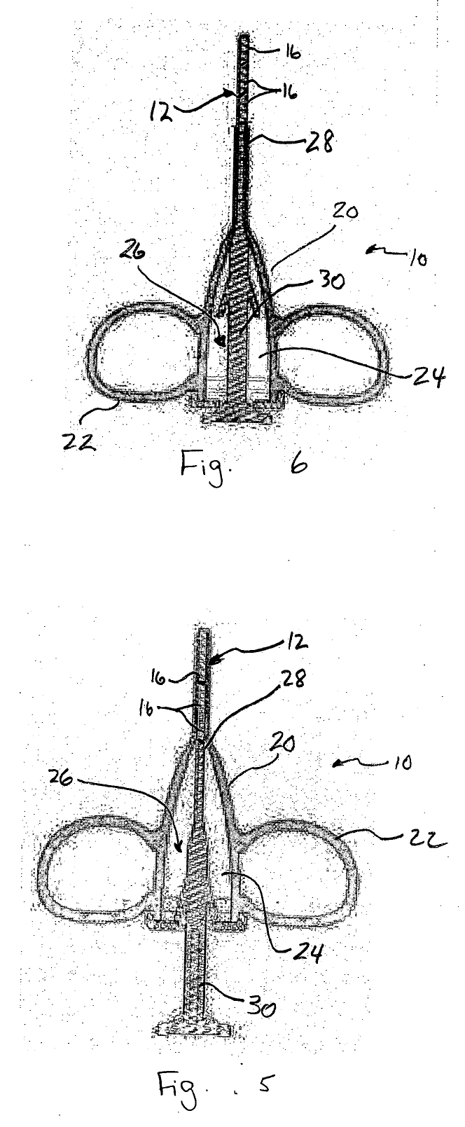

FIG. 5 depicts one embodiment of suitable delivery device or instrument 10 for use in the method of the present invention. The device 10 comprises a body 20 having appropriate or suitable gripping means 22 associated with it in some embodiments. The body 20 includes a central bore or hollow region 24 housing an injector structure 26 including, at one of the body 20, a lumen 28. As shown in FIG. 5, the lumen 28 contains a plug body 12 comprised of a selected number of segments 16 in preparation for being injected into a tissue tract.

FIG. 6 depicts the embodiment of the delivery device 10 shown in FIG. 5 wherein a plunger 30 is axially moved toward patient (not shown) to eject or displace the plug 12 from the lumen 28 of the device 10 into a tissue tract (not shown).

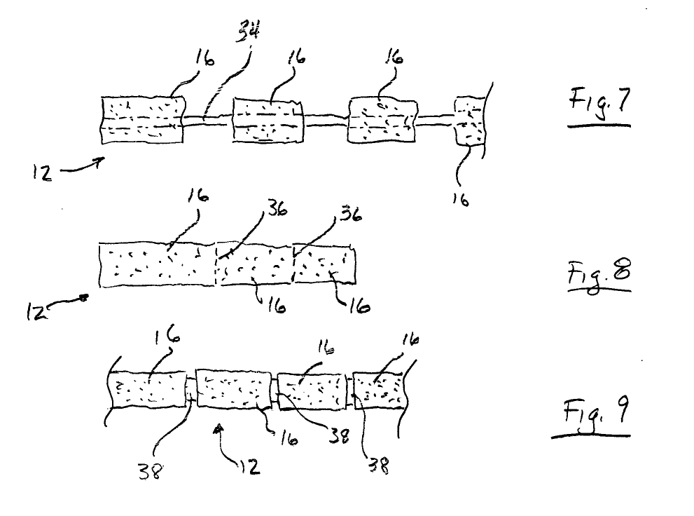

FIGS. 7-9 depict additional embodiments of a tract plug 12 in accordance with the present invention. As in embodiments of the present invention, the embodiments of FIGS. 7-9 may be formed entirely or partially from suitable material, including hemostasis promoting material. FIG. 7 depicts an embodiment of the invention wherein a series of individual segments or units 16 of a plug body 12 are separably coupled to one another by means of an elongated, flexible member 34. The member 34 may be formed of the same material as the individual segments 16 or it can be formed from another suitable material, including a hemostasis promoting material. This embodiment may be thought of as a plurality of individual beads 16 of collagen which are removably received on a string, i.e., the elongated member 34. In use, the selected number of individual beads or segments 16 of collagen could be loaded into the lumen 28 of a delivery device 10 on the string 34 or stripped from the string 34 serially or sequentially into the lumen 10. Alternatively, they could be delivered into a tract while the elongated member 34 is in place and, once the position of the plug 12 is satisfactory, the elongated member 34 could simply be withdrawn from the tract leaving the collagen beads 16 in place abutted against each other surface-to-surface or end-to-end filling the tract. Any bead 16 or portion thereof remaining outside the skin of the patient may simply be pulled, broken away or separated from those beneath skin level in the tract (as depicted in FIG. 1c). In some embodiments, the elongated member, the string, a tab or like suitable structure, or a portion thereof, can be pulled, twisted or otherwise manipulated to separate segments or to initiate segment separation.

FIG. 8 depicts an embodiment of a plug body 12 in accordance with the present invention wherein the collagen segments 16 are joined end-to-end over or across a line of weakness 36. In the depicted embodiment, the line of weakness 36, which can be formed in any suitable way, comprises a plurality of perforations, short cuts or spots of weakness. In use, the plug 12, comprising a suitable number of individual segments 16 connected to each other, would be placed in a suitable delivery device or into the tract, and if the outermost segment 16 extended beyond the skin “S” of the patient as depicted in FIG. 1b, it would simply be grasped and separated along the line of weakness 36 from the remaining individual segments 16.

FIG. 9 depicts another embodiment of a plug body 12 in accordance with the present invention wherein the individual segments 16 of a hemostasis promoting material are connected or joined by a breakable portion 38 comprising the same or other suitable material. In use, the overall length of the plug 12 can be selected by selecting the number of plug segments 16 by snapping, tearing or breaking the connective portion 38 extending between the individual segments 16.

In use, a suitable delivery device 10 such as that depicted or represented in the Figures is used to deliver a segmented collagen plug 12 into a tissue tract to effect hemostasis. The collagen plug 12 is a composite of or comprises several smaller collagen segments or members 16 in immediate contact, end-to-end contact, with each other. The collagen plug 12 may be placed in the lumen 28 of a delivery device 10 for delivery into the tissue tract as depicted in FIGS. 1a-c. The collagen plug body 12 may comprise initially physically separated segments 16 which are placed in end-to-end contact and adhere to each other by virtue of their physical makeup. For example, if a fibrous segment 16 is used and is placed into contact with another similar or different fibrous segment 16, the two segments 16 will adhere to each other because of entanglement of the fibers on the ends of the segments 16, much the way connectors known as hook and loop connective fabric are connectable and separable. Also, as shown in FIGS. 7-9 other connective structures may be utilized. After being inserted into a tissue tract or wound, the depth or length of which may vary, some or none of the collagen segments 16 may remain above skin level. Any segment 16 appearing entirely or partially above skin level may be removed easily, without cutting, as depicted in FIG. 1c, while leaving the remaining segments 16 in the tract below skin level. The advantageous and desired effect is to have an adjustable length of collagen plug 12 without requiring a physician or physician's assistant to pre-measure the length of the tract or cut the plug 12 to length after it is placed in the tract. The extra collagen segments 16′, after being torn or pulled free, may simply be discarded. Thus, there is no need to premeasure the tract, cut off the exposed collagen, or to include a depth marker on the delivery device 10.

While a collagen tract plug 12 in accordance with the present invention is depicted in three or four segments 16, it should be appreciated that any number of any individual segments or units 16 may be used to form a tract plug body 12 in accordance with the present invention. Any suitable material, including hemostasis promoting material, may be used, and the plug 12 may have any suitable configuration or size.

In one embodiment, the collagen plug 12 may be compressed prior to delivery into a tract “T”. In this instance, with reference to FIG. 5, the collagen would be compressed as or after it is inserted into the lumen 28 of a delivery device 10 and would expand after it is ejected from the device 10 into a tract upon being exposed to blood. FIG. 4 depicts the expandable nature of collagen, namely that a collagen plug of the general size depicted in FIGS. 2 and 3 may be expanded by being wetted once in a tract.

Although the present invention has been described with reference to preferred embodiments, persons skilled in the art will recognize that changes may be made in form and detail without departing from the spirit and scope of the invention.

1. A tract plug comprising segments of biosorbable material wherein the segments are linked.

2. The tract plug according to claim 1, wherein the segments are severably linked.

3. The tract plug according to claim 2, wherein the segments are linked across a line of weakness.

4. The tract plug according to claim 3, wherein the line of weakness comprises perforations.

5. The tract plug according to claim 3, wherein the plug has a length and the line of weakness is generally transverse to the length, whereby when the segments are separated from each other along the line of weakness the length is shortened.

6. The tract plug according to claim 2, wherein the segments comprise fibers and said linking is accomplished by contact between the fibers of one segment with the fibers of another segment.

7. The tract plug according to claim 6, wherein said contact is created by compression.

8. The tract plug according to claim 7, wherein the biosorbable material is collagen.

9. The tract plug according to claim 1, and a device for inserting the tract plug into a tract.

10. A generally elongated body comprising a biocompatible material, wherein the body comprises a number of units comprising a biocompatible material, wherein the units are separably joined so that the length of the body may be selected initially by joining a selected number of the units to form the body and after the body is formed by separating one or more of the joined units from the body.

11. The body according to claim 10, wherein the biocompatible material comprises a hemostasis promoting material.

12. The body according to claim 11, wherein the hemostasis promoting material is collagen.

13. The body according to claim 11, wherein the units are joined by contacting one unit with another.

14. A method of substantially filling a tissue tract comprising the steps of:
providing a tract plug comprising segments;
inserting the tract plug into the tissue tract; and
separating any segment remaining outside the tract from the segments in the tissue tract after the tract plug is inserted.

15. The method according to claim 14, wherein the segments are linked.

16. The method according to claim 15, wherein the link between the segments is breakable.

17. The method according to claim 14, further comprising the step of loading the segments of the tract plug into a delivery device and using the delivery device to insert the tract plug into the tract.

18. The method according to claim 17, wherein the segments comprise collagen.

19. The method according to claim 17, wherein the tract plug is inserted in the tract such that an end of the tract plug is generally adjacent to a previously placed vascular closure.
